SEQUENCE LISTING (5 sequences)

SEQ ID NO: 1 - protein, 773 aa, Bacillus sp.
AEGNTREDNFKHLLGNDNVKRPSEAGALQLQEVDGQMTLVDQHGEKIQLRGMSTHGLQWFPEILNDNAYKALANDWESNMIRLAMYVGENGYASNPELIKSRVIKGIDLAIENDMYVIVDWHVHAPGDPRDPVYAGAEDFFRDIAALYPNNPHIIYELANEPSSNNNGGAGIPNNEEGWNAVKEYADPIVEMLRDSGNADDNIIIVGSPNWSQRPDLAADNPINDHHTMYTVHFYTGSHAASTESYPPETPNSERGNVMSNTRYALENGVAVFATEWGTSQANGDGGPYFDEADVWIEFLNENNISWANWSLTNKNEVSGAFTPFELGKSNATNLDPGPDHVWAPEELSLSGEYVRARIKGVNYEPIDRTKYTKVLWDFNDGTKQGFGVNSDSPNKELIAVDNENNTLKVSGLDVSNDVSDGNFWANARLSADGWGKSVDILGAEKLTMDVIVDEPTTVAIAAIPQSSKSGWANPERAVRVNAEDFVQQTDGKYKAGLTITGEDAPNLKNIAFHEEDNNMNNIILFVGTDAADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVAFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTEPVEPEPVDPGEETPPVDEKEAKKEQKEAEKEEKEE

SEQ ID NO: 2 - protein, 294 aa, Sordaria fimicola
MRSSTILQTGLVAVLPFAVQAASGSGKSTRYWDCCKPSCAWSGKASVNRPVLACDANNNPLNDANVKSGCDGGSAYTCANNSPWAVNDNLAYGFAATKLSGGTESSWCCACYALTFTSGPVSGKTLVVQSTSTGGDLGSNHFDLNMPGGGVGLFDGCKREFGGLPGAQYGGISSRSECDSFPAALKPGCQWRFDWFKNADNPEFTFKQVQCPSELTSRTGCKRNDDSQFPAFTPPSGGGSNPSTPTTPPSSGGGGSGCAAAMYAQCGGSGFSGCTNCPSGSTCKAINDYYHQCA

SEQ ID NO: 3 - protein, 305 aa, Humicola insolens
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPSSSTSSPVNQPTSTSTTSTSTTSSPPVQPTTPSGCTAERWAQCGGNGWSGCTTCVAGSTCTKINDWYHQCL

SEQ ID NO: 4 - protein, 299 aa, Thielavia terrestris
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDSSFPVFTPPSGGNGGTGTPTSTAPGSGQTSPGGGSGCTSQKWAQCGGIGFSGCTTCVSGTTCQKLNDYYSQCL

SEQ ID NO: 5 - protein, 295 aa, Staphylotrichum coccosporum
ADGKSTRYWDCCKPSCSWPGKASVNQPVFACSANFQRISDPNVKSGCDGGSAYACADQTPWAVNDNFSYGFAATSISGGNEASWCCGCYELTFTSGPVAGKTMVVQSTSTGGDLGTNHFDLAMPGGGVGIFDGCSPQFGGLAGDRYGGVSSRSQCDSFPAALKPGCYWRFDWFKNADNPTFTFRQVQCPSELVARTGCRRNDDGNFPVFTPPSGGQSSSSSSSSSAKPTSTSTSTTSTKATSTTSTASSQTSSSTGGGCAAQRWAQCGGIGFSGCTTCVSGTTCNKQNDWYSQCL

(19) United States
(12) Patent Application Publication
(10) Pub. No.: US 2020/0277553 A1
(43) Pub. Date: Sep. 3, 2020

(54) Use of Enzymes for Improving Water Absorption And/Or Whiteness

(71) Applicant: Novozymes A/S, Bagsværd (DK)

(72) Inventors: Elena Genesca PONT, Kobenhavn (DK); Bitten PLESNER, Copenhagen (DK); Tina Reenberg HANSEN, Sverige (DK); Janni Stentz Estholm KEPPIE, Greve (DK)

(73) Assignee: Novozymes A/S, Bagsværd (DK)

(21) Appl. No.: 16/645,846

(22) PCT Filed: Sep. 19, 2018
(86) PCT No.: PCT/EP2018/075328

§ 371 Date: Mar. 10, 2020

(30) Foreign Application Priority Data
Sep. 20, 2017 (EP) 17192111.7

(51) Int. Cl. C11D 3/386; C12N 9/42; C11D 3/00

(52) U.S. Cl. CPC C11D 3/38645; C12Y 302/01004; C11D 3/001; C12N 9/2437

(57) ABSTRACT

The present invention concerns the use of enzymes for improving water absorption and/or improving whiteness of a textile. The invention further concerns a softener composition for use in improvement of water absorption and/or improvement of whiteness of a textile.

### FIELD OF THE INVENTION

The present invention concerns the use of enzymes, in particular cellulases, for improving water absorption and/or improving whiteness of a surface, such as a textile, wherein the use is in a softener.

### REFERENCE TO A SEQUENCE LISTING

This application contains a Sequence Listing in computer readable form. The computer readable form is incorporated herein by reference.

### BACKGROUND OF THE INVENTION

Use of enzymes in laundry detergents is well known. Also use of enzymes capable for degrading cellulosic material is known for laundry purpose. However, cellulose degrading enzymes for laundry should be selected carefully as laundry textile serve as substrate for the enzymes.

The degradation of cellulosic material in washing machines is often a challenge. Cellulosic fibers may be cleaved from textile during wash with enzymes capable for degrading cellulosic material and tend to clog filters, pipes and drains in washing machines. The drains and filters thus need to be cleaned manually from time to time.

Wearing, washing and tumble drying of fabric and textile exposes the textile to mechanical stress which damages the textile and fabric by breaking the fibers in the fabric/textile and thereby causing the textile/fabric to be covered with fuzz and pills. This gives the fabric or textile a worn look.

It has been known to use cellulases and other enzymes in laundry detergents. Softeners are often used to make the feel of the clothes smoother/softer. However, as the function of softeners is to coat the surface of a fabric with chemical compounds that are electrically charged, this may limit e.g. a towel's properties to absorb water. Thus, there is a need to for improvement of water absorption without compromising the softeners properties of making fabrics feel smooth and soft.

### SUMMARY OF THE INVENTION

In one aspect, the present invention relates to the use of an enzyme for improving water absorption of a textile by adding an enzyme to a softener.

In another aspect, the present invention also relates to the use of an enzyme for improving whiteness of a textile by adding an enzyme to a softener.

In another aspect, the present invention relates to a softener composition for use in improving water absorption and/or whiteness of a textile, wherein said softener composition comprises a family GH45 cellulase, preferably a cellulase having at least 60% sequence identity to SEQ ID NO: 1, 2, 3, 4 or 5.

### Definitions

Anti-pilling: The term “anti-pilling” denotes removal of pills from the textile surface and/or prevention of formation of pills on the textile surface.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (chose, 1987, Measurement of cellulase activities, Pure Appl. Chem. 59: 257-68).

For purposes of the present invention, cellulolytic enzyme activity is determined by measuring the increase in hydrolysis of a cellulosic material by cellulolytic enzyme(s) under the following conditions: 1-50 mg of cellulolytic enzyme protein/g of cellulose in PCS (or other pretreated cellulosic material) for 3-7 days at a suitable temperature, e.g., 50° C., 55° C., or 60° C., compared to a control hydrolysis without addition of cellulolytic enzyme protein. Typical conditions are 1 ml reactions, washed or unwashed PCS, 5% insoluble solids, 50 mM sodium acetate pH 5, 1 mM MnSO₄, 50° C., 55° C., or 60° C., 72 hours, sugar analysis by AMINEX® HPX-87H column (Bio-Rad Laboratories, Inc., Hercules, Calif., USA).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in vegetable food products, such as salad, tomatoes, spinach, cabbage, grain or the like.

Detergent component: The term “detergent component” is defined herein to mean the types of chemicals which can be used in detergent compositions for laundry. Examples of detergent components are surfactants, builders, chelators or chelating agents, bleach system or bleach components, polymers, fabric conditioners, foam boosters, suds suppressors, dyes, perfume, tannish inhibitors, bactericides, fungicides, soil suspending agents, anti-corrosion agents, enzyme inhibitors or stabilizers, enzyme activators, transferase(s), hydrolytic enzymes, oxido reductases, blueing agents and fluorescent dyes, antioxidants, and solubilizers.

Detergent Composition: The term “detergent composition” refers to compositions that find use in the removal of undesired compounds from surfaces to be cleaned, such as textile surfaces. The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). The detergent composition may contain one or more enzymes such as hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, beta-glucanases, arabinosidases, hyaluronidase, chondroitinase, laccase, DNase, chlorophyllases, amylases, perhydrolases, peroxidases, xanthanase and mixtures thereof. The detergent composition may further comprise detergent component such as surfactants, builders, chelators or chelating agents, bleach system or bleach components, polymers, fabric conditioners, foam boosters, suds suppressors, dyes, perfume, tannish inhibitors, bactericides, fungicides, soil suspending agents, anti-corrosion agents, enzyme inhibitors or stabilizers, enzyme activators, transferase(s), hydrolytic enzymes, oxido reductases, blueing agents and fluorescent dyes, antioxidants, and solubilizers.

Fabric softener: A Fabric softener (also called fabric conditioner or solely softener) is a composition that is typically applied to laundry during the rinse cycle in a washing machine or when washing by hand. Fabric softeners are available as solutions and solids, and may also be impregnated in dryer sheets used in a clothes dryer.

Fabric softener agent: A fabric softener agent (or a softener agent) is an ingredient that is comprised in fabric softener compositions such as chemical compounds that are electrically charged. These compounds causes threads in the fabric to lift up from the surface of the textile and thereby gives the fabric a softer feel of the textile. In one embodiment the fabric softener agent is one ore more cationic softeners. The cationic softeners bind by electrostatic attraction to the negatively charged groups on the surface of the textile and neutralize their charge and thereby impart lubricity.

Fragment: The term “fragment” means a polypeptide having one or more (e.g., several) amino acids absent from the amino and/or carboxyl terminus of a mature polypeptide main; wherein the fragment has enzyme activity. In one aspect, a fragment contains at least 85%, e.g., at least 90% or at least 95% of the amino acid residues of the mature polypeptide of an enzyme.

Hemicellulolytic enzyme or hemicellulase: The term “hemicellulolytic enzyme” or “hemicellulase” means one or more (e.g., several) enzymes that hydrolyze a hemicellulosic material. See, for example, Shallom, D. and Shoham, Y. Microbial hemicellulases. Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates of these enzymes, the hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature, e.g., 50° C., 55° C., or 60° C., and pH, e.g., 5.0 or 5.5.

Family GH45 cellulase: the term “family GH45 cellulase” as used herein, refers to Glycosyl hydrolases are enzymes that catalyze the hydrolysis of the glycosyl bond. There are over 100 classes of Glycosyl hydrolases which have been classified, see Henrissat et al. (1991) A classification of glycosyl hydrolases based on amino-acid sequence similarities', J. Biochem. 280: 309-316 and the CAZY website at www.cazy.org. The glycoside hydrolases of family 45 (GH45) have so far been identified as endoglucanase (EC 3.2.1.4). Within the definition falls enzymes which are commonly known as “cellulases”. Such enzymes comprises also enzymes that may be known as endoglucananses.

Rinse cycle: The term “rinse cycle” is defined herein as a rinsing operation wherein textile is exposed to water for a period of time by circulating the water and optionally mechanically treat the textile in order to rinse the textile and finally the superfluous water is removed. A rinse cycle may be repeated one, two, three, four, five or even six times at the same or at different temperatures.

Whiteness: The term “Whiteness” is defined herein as a broad term with different meanings in different regions and for different consumers. Loss of whiteness can e.g. be due to greying, yellowing, or removal of optical brighteners/hueing agents. Greying and yellowing can be due to soil redeposition, body soils, colouring from e.g. iron and copper ions or dye transfer. Whiteness might include one or several issues from the list below: colourant or dye effects; incomplete stain removal (e.g. body soils, sebum etc.); redeposition (removed soils reassociate with other parts of textile, soiled or unsoiled); chemical changes in textile during application; and clarification or brightening of colours.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Softener: The term “softener” as used herein refers to a composition used, in particular, in laundry settings. A softener is primarily used in the rinse step in the laundry process where the softener is added to the rinse water after the washing with a laundry detergent. Fabric softeners coat the surface of a fabric with chemical compounds that are electrically charged, neutralizing the charge of the fabric and causing threads to “stand up” from the surface so the fabric feels softer and makes it fluffier.

Variant: The term “variant” means a polypeptide having enzyme activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Wash cycle: The term “wash cycle” is defined herein as a washing operation wherein textile is exposed to the wash liquor for a period of time by circulating the wash liquor and textile in a washing machine. A wash cycle may be repeated one, two, three, four, five or even six times at the same or at different temperatures. The wash cycle if often followed by a rinse cycle and finally a centrifugation cycle where water is removed from the textile. It is known for the skilled person to determine which is the wash cycle during laundry wash.

Wash liquor: The term “wash liquor” is intended to mean the solution or mixture of water and detergents optionally including enzymes used for laundry.

### DETAILED DESCRIPTION OF THE INVENTION

The present invention relates to the use of an enzyme for improving water absorption of a textile by adding the enzyme to a softener. The invention also relates to a method for improving water absorption of a textile comprising contacting a surface, such as a fabric surface, with an enzyme and a softener. The present inventors have found that by adding an enzyme to a softener, the water absorption is improved as compared to when using a softener without an enzyme. A softener is typically applied to laundry during the rinse cycle in a washing machine. Typically, fabric softeners are available as solutions and solids, and may also be permeated in dryer sheets used in a clothes dryer.

When improving the water absorption of a textile, it has the benefit that items such as towels, can absorb more water when used for drying a skin or surfaces.

The present invention also relates to the use of an enzyme for improving whiteness of a textile by adding said enzyme to a softener. The invention relates to a method for improving whiteness of a textile comprising contacting a surface, such as a fabric surface, with an enzyme and a softener. White clothes often turn into a greyish shade upon usage and repeatedly washes. The inventors of the present invention have found that by addition of an enzyme to the softener, the whiteness of clothes can be maintained as compared to using a softener without an enzyme.

Use of enzymes for washing surfaces are commonly know. E.g. cellulases has been used in laundry detergent for a long time in order to remove fuzz and pills on the fabric surface.

In a particular embodiment, the enzyme used in the softener is a family GH45 cellulase.

It has not previously been shown that using a family GH45 cellulase in softeners can improve the water absorbtion and/or whiteness of a fabric. As can be seen in the examples of the present invention, both water absorbtion and whiteness are improved when a cellulase has been added to the softener.

In one of the examples, the water absorption has been evaluated as water level (cm) after 1 hr, wherein the textile has been vertically put in a beaker. In a further embodiment, the assay comprises the step of pre-washing the textile multiple times before evaluation of water absorbtion, and optionally, the textile has been tumble dried in-between each wash.

In another example, the whiteness of a fabric is measured by absorbance defined by remission at 460 nm. In a further embodiment, the assay comprises the step of pre-washing the textile multiple times before evaluation of whiteness, and optionally, the textile has been tumble dried in-between each wash.

In one embodiment, the enzyme is a cellulase having at least 60% sequence identity to SEQ ID NO: 1, 2, 3, 4, or 5.

The cellulase may be any one having at least 60% sequence identity to SEQ ID NO: 1, SEQ ID NO: 2, SEQ ID NO: 3, SEQ ID NO: 4, or SEQ ID NO: 5, preferably the cellulase has at least 65%, such as 70%, such as 75%, such as 80%, such as 85%, such as 90%, such as 91%, such as 92%, such as 93%, such as 94%, such as 95%, such as 96%, such as 97%, such as 98%, such as 99%, or such as 100%, sequence identity to SEQ ID NO: 1, SEQ ID NO: 2, SEQ ID NO: 3, SEQ ID NO: 4, or SEQ ID NO: 5, or a fragment thereof having cellulase activity.

A softener may also be termed “fabric softener” or even “fabric conditioner” and the components of such a softener, may differ in affinity to various fabrics. Some work better on cellulose-based fibers (i.e., cotton), others have higher affinity to hydrophobic materials like nylon, polyethylene terephthalate, polyacrylonitrile, etc. Other silicone-based compounds, such as polydimethylsiloxane, work by lubricating the fibers. Derivatives with amine- or amide-containing functional groups may be included as well. These groups improve the softener's binding to fabrics.

As softeners are often hydrophobic, they commonly occur in the form of an emulsion. In the early formulations, manufactures used soaps as emulsifiers. The emulsions are usually opaque, milky fluids. However, there are also microemulsions, where the droplets of the hydrophobic phase may be substantially smaller. Microemulsions provide the advantage of increased ability of smaller particles to penetrate into the fibers.

The softener may be a mixture of cationic and non-ionic surfactants as an emulsifier. Another approach is a polymeric network, an emulsion polymer.

In one embodiment, the softener comprises cationic surfactants, such as esterquats. Characteristically, the cations contain one or two long alkyl chains derived from fatty acids. Other cationic compounds can be derived from imidazolium, substituted amine salts, or quaternary alkoxy ammonium salts.

It is believed that cationic surfactants may have a beneficial effect on the enzyme's ability to improve the water absorbtion and/or whiteness. It is hypothesized that the surfactant level in a softener may be lowered when an enzyme is added to the softener. This will have a beneficial effect on the environment as surfactants can be harsh on the environment.

In one embodiment, the softener has a pH of at least 2.0, such as at least 2.4, such as at least 3.0. The softener to which the enzyme is added typically has a pH of 2.0 to 5.0, preferably in the range of 2.4 to 4.5, or even more preferred in the range of 3.0 to 3.5. Thus, the enzyme that is added to the softener is an enzyme that is stable at such pH. When the composition, such as the softener, to which the enzyme is added as a pH which is within the optimal pH range of the enzyme, said pH will not affect the enzyme in a negative way. Therefore, it is believed that the pH of the softener and the enzyme complement each other in their function. Thus, the enzyme will provide the whiteness and/or improved water absorption, whereas the pH will make sure that the surfactant works and bring softness to the treated fabric.

In one embodiment, the textile which has improved water absorption and/or whiteness when rinsed with a softener comprising an enzyme, the textile has been pre-washed in a laundering process.

Often when laundering textile, such as clothes, the wash cycle comprises both a wetting step, i.e. where water is let in to the machine and the textile thereby gets wet, a washing step, i.e. where the laundry detergent is added to the washing liquid, a rinse step, i.e. where optionally a softener is added to the rinse liquid, and finally a centrifugation step, i.e. where the textile is centrifuged in order to relieve the textile for as much water as possible before the textile is dried.

In one embodiment, the textile is cotton, polyester, or a mixture thereof.

The textile may be any pure form, such as 100% cotton, 100% polyester or the like, or it may be any blend of different types of textile, such as 50% cotton and 50% polyester. Thus, in one embodiment, the textile is a mixture of at least 50% polyester and at least 20% cotton.

In another embodiment, the textile is cotton.

The laundering process may be done at various temperatures depending on the textile, the level of dirt on the textile, or any other aspect that may be dependent on the temperature. The invention is not limited to any specific temperature. Thus, in one embodiment, the pre-washing has been done at a temperature of at least 5° C., such as at least 10° C., at least 15° C., at least 20° C., at least 25° C., at least 30° C., at least 35° C., at least 40° C., at least 45° C., or at least 50° C.

The concentration of the enzyme added to the softener may vary, but in one embodiment, the enzyme is added in a concentration of at least 0.01% of said softener.

In another aspect, the invention also relates to a softener composition for use in improving water absorption and/or whiteness of a textile, wherein said softener composition comprises a family GH 45 cellulase, preferably a cellulase having at least 60% sequence identity to SEQ ID NO: 1, 2, 3, 4, or 5.

The softener composition may further comprise a preservative and/or biocide. The preservative and/or biocide is selected from metholisothiazolinone or methylchlorisothiazolinone or a combination of metholisothiazolinone and methylchlorisothiazolinone. Metholisothiazolinone and methylchlorisothiazolinone have preserving effect and biocidal effect.

In the case of a liquid softener composition, adding an acid to the softener composition enables water-soluble metal salts to at least partially dissolve in the composition. The acid also helps to at least partially reduce the precipitation on hard surfaces during the rinse cycle. The acid may also stabilize the liquid softener composition against precipitation in the product prior to use.

In the case of a solid softener composition, adding an acid to the softener composition enables water-soluble metal salts, once released, to at least partially dissolve quickly in the wash and/or rinse liquor of a laundry appliance so as to prevent insoluble material from forming and/or from depositing onto the surfaces, such as on textile.

In one embodiment more than one enzyme may be added to the softener, and thus, in addition to the at least one enzyme used in the improvement of water absorbtion and/or whiteness of the textile. The one or more enzymes may be selected from the group consisting of amylases, hemicellulases, peroxidases, proteases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, R-glucanases, arabinosidases, hyaluronidase, chondroitinase, laccase, DNase chlorophyllases, amylases, perhydrolases, peroxidases, proteases, xanthanase and mixtures thereof. The enzymes are described in further details below.

Suitable amylases which can be used in the rinse aid composition of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions: M197T; H156Y+A181T+N190F+A209V+Q264S; or G48A+T491+G107A+H156Y+A181T+N190F+I201F+A209V+0264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Other amylases are variants of SEQ ID NO: 1 of WO 2016/203064 having at least 75% sequence identity to SEQ ID NO: 1 thereof. Preferred variants are variants comprising a modification in one or more positions corresponding to positions 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 of SEQ ID NO: 1, wherein said alpha-amylase variant has a sequence identity of at least 75% but less than 100% to SEQ ID NO: 1.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T1311, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+1182G+F202Y+Y305R+D319T+G475K;

S125A+N128C+K178L+T182G+Y305R+G475K; or

S125A+N128C+T1311+T1651+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K

wherein the variants optionally further comprises a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128, K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

N21D+D97N+V128I

wherein the variants optionally further comprises a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme PIus™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the 51 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus Lentus protease shown in SEQ ID NO 1 of WO 2016/001449. More preferred the subtilase variants may comprise the mutations: S3T, V41, S9R, S9E, A15T, S24G, S24R, K27R, N42R, S55P, G59E, G59D, N60D, N60E, V66A, N74D, N85S, N85R, G96S, G96A, S97G, S97D, S97A, S97SD, S99E, S99D, S99G, S99M, S99N, S99R, S99H, S101A, V1021, V102Y, V102N, S104A, G116V, G116R, H118D, H118N, N120S, S126L, P127Q, S128A, S154D, A156E, G157D, G157P, S158E, Y161A, R164S, Q176E, N179E, S182E, Q185N, A188P, G189E, V193M, N198D, V1991, Y203W, S206G, L211Q, L211D, N212D, N212S, M216S, A226V, K229L, Q230H, Q239R, N246K, N255W, N255D, N255E, L256E, L2560 T268A, R269H. The protease variants are preferably variants of the Bacillus Lentus protease (Savinase®) shown in SEQ ID NO 1 of WO 2016/001449, the Bacillus amylolichenifaciens protease (BPN′) shown in SEQ ID NO 2 of WO2016/001449. The protease variants preferably have at least 80% sequence identity to SEQ ID NO 1 or SEQ ID NO 2 of WO 2016/001449.

A protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes A/S), those sold under the tradename Maxatase®, Maxacal®, Maxapem®, Purafect Ox®, Purafect OxP®, Puramax®, FN2®, FN3®, FN4®, Excellase®, Excellenz P1000T™, Excellenz P1250™, Eraser®, Preferenz P100™, Purafect Prime®, Preferenz P110™, Effectenz P1000™, Purafect®™, Effectenz P1050T™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes NS) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation), Revitalenz® 200 (Danisco/Dupont), and Revitalenz® 2000 (Danisco/Dupont).

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes NS), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase according to the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Concentration of the Enzyme

In one embodiment, the enzyme added to the softener may be used in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The enzyme(s) of the softener composition of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708.

A polypeptide of the present invention may also be incorporated in the detergent formulations disclosed in WO97/07202, which is hereby incorporated by reference.

### Surfactants

The softener composition may comprise one or more surfactants, which may be cationic and/or non-ionic.

When included therein, the softener will usually comprise from about from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include bis(Acyloxyethyl)hydroxyethyl Methylammonium Methosulphate, Dipalmoylethyl hydroxyethylmonium methosulfate, dihydrogenated tallow hydroxyethylmonium methosulfate, distearoylethyl hydroxyethylmonium methosulfate, dioleoyl ethyl hydroxyethylmonium methosulfate alkyl quaternary ammonium compounds, alkoxylated quaternary ammonium (AQA) compounds, other ester quats, and combinations thereof.

When included therein, the softener will usually comprise from about 0.1% to about 10% by weight of a nonionic surfactant, for example from about 0.2% to about 5%, in particular from about 0.2%% to about 3%, such as from about 0.2% to about 0.5%, from about 0.5% to about 1%, or from about 1% to about 3%. Non-limiting examples of nonionic surfactants include polysorbates, polyethylene glycol ethers, Polyoxyethylene alkyl ethers, alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

### Builders and Co-Builders

The softener composition may comprise about 0-10% by weight, such as about 0.1% to about 5% of a builder or co-builder, or a mixture thereof. In a softener, the level of builder is typically 0-1%, particularly 0-0.5%. The builder and/or co-builder may particularly be a chelating agent that forms water-soluble complexes with Ca and Mg. Any builder and/or co-builder known in the art for use in softener may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The softener composition may also comprise 0-5% by weight, such as about 0% to about 2%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N,′N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Polymers

The softener may comprise 0-10% by weight, such as 0.5-5%, 2-5%, 0.5-2% or 0.2-1% of a polymer. Any polymer known in the art for use in softeners may be utilized. The polymer may function as a co-builder as mentioned above, or may provide antiredeposition, fiber protection, soil release, dye transfer inhibition, anti-foaming properties, perfume encapsulation and lubricity.

Some polymers may have more than one of the above-mentioned properties and/or more than one of the below-mentioned motifs. Exemplary polymers include polyquaterniums, melamine polymers, siloxanes, silicones, carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC), copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Perfume

The softener compositions may comprise a perfume in a free form or encapsulated. The perfume composition may comprise perfume ingredients such as, but not exclusively, butylphenyl methylpropional, geraniol, benzyl salicylate, hexyl cinnamal, amyl cinnamal, limonene, benzisothiazolinone, alpha isomethyl ionone, linalool. Any fragrance, perfume or perfume oil known in the art for use in softeners may be utilized

### Adjunct Materials

Any softener component known in the art for use in softeners may also be utilized. Other optional softener components include solvents (including isopropyl alcohol, propylene glycol, alkane/cycloalkane), anti-shrink agents, anti-soil redeposition agents, anti-wrinkling agents, bactericides, preservatives (including benzisothiazolinone, methylisothiazolinone and/or lactic acid), binders, dyes, enzyme stabilizers (including boric acid, borates, CMC, and/or polyols such as propylene glycol), emulsion stabilizers, antifoam agents (including dimethicone), skin conditioning agents (including caprylic/capric glycerides, ethylhexyl stearate, or cocos oil, either alone or in combination. Any ingredient known in the art for use in softeners may be utilized. The choice of such ingredients is well within the skill of the artisan.

The present invention is further described by the following examples that should not be construed as limiting the scope of the invention.

### EXAMPLES

### Detergent

### Composition of Model Detergent A:

| Ingredient | Content (% w/w) |
|---|---|
| (C10-C13)alkylbenzene sulfonic acid | 12 |
| Sodium lauryl ether sulfate | 5 |
| Soy fatty acid | 2.75 |
| Coco fatty acid | 2.75 |
| Alcohol ethoxylate | 11 |
| Sodium hydroxide | 2 |
| Ethanol | 3 |
| Propane-2-ol | 0.3 |
| Propane-1,2-diol | 6 |
| Glycerol | 2 |
| Triethanolamine | 3 |
| Sodium formate | 1 |
| Sodium citrate | 2 |
| DTMPA-Na7 | 0.2 |
| Copoly(acrylic acid/maleic acid), sodium salt | 0.2 |
| Phenoxyethanol | 0.5 |

### Example 1—Water Absorption Improvement Evaluation

### Procedure for Preparing the Textiles

### Enzymes Used:

Cellulase (SEQ ID NO: 4) available from Novozymes A/S, Bagsvaerd, Denmark.

### Detergent and Softener Used:

Detergent: Neutral, Denmark (Unilever). Water, C12-12 Pareth-7, Sodium Laureth Sulfate, Alcohol, Potassium Cocoate, Potassium Citrate, Trietanolamine, Sodium Diethylenetriamine Pentamethylene Phosphonate, glycerin, PVP, Propylene glycol, Calcium chloride, Potassium hydroxide, Protease, Process by products (Peptides, salts sugars from fermentation), Boronic acid (4-formylphenil), Amylase, Sodium chloride, C11-15 Sec-Pareth-12.

Softener: Ideel, Denmark (Aldi). Water, Cationic surfactants, Isopropyl alcohol, Calcium Chloride.

### Textiles Used:

W-10A (WFK standard cotton, 50×1 m), W-20A (WFK Polyester/Cotton 65/35%, 50×1 m), W-30A (WFK 100% Polyester, 50×1 m).

### Machine Used:

Miele Softronic W3241.

### Washing Conditions:

Wash temperature: 40° C., short program (1 h 35 min), Water hardness: 15 dH,

Water level (in wash): 13-14 L water (main wash), Ballast: Total of 3 kg, Spinning speed: 1600 rpm

Tumble drying: the Miele ECO TCE 630 WP tumble dryer was used with the programe “Cotton—skabstørt” (Cotton—Cupboard dry).

Procedure: One Sheet of Each of the Materials was Added to the Wash and 20 consecutive cycles were performed in the defined washing conditions with tumble drying in between each of the washes and after the last wash. Water absorption was measured after the 20 cycles of wash and tumble drying. Results can be seen in Table 1 below.

### Procedure for Evaluating the Water Absortion of the Textiles:

Textiles were evaluated according to the vertical wicking test.
1. For each textile 5 2×20 cm stripes of textile were cut.
2. For each evaluation 20 ml of deionized water with food colorant (20 drops per 100 ml) were added in two 8 cm diameter beakers.
3. One stripe treated with softener and one stripe treated with softener with the cellulase of SEQ ID NO: 4 were put vertically in one of the beakers. One end was under the water level and the other end was attached to a support to ensure the stripes were absolutely vertical.
4. The height of the water level after 1 hour was measured.
5. The evaluation was done by quintuplicate (5 stripes).

TABLE 1
Results showing the water absorption improvement
when using a cellulase in a softener

| | | | Water level |
|---|---|---|---|
| | No. | | (cm) after |
| Textile | repetitions | | 1 h |
| W10A | 1 | Softener | 11.4 |
| | 1 | Softener + 0.25% Cellulase of | 11.8 |
| | | SEQ ID NO: 4 | |
| | 2 | Softener | 11.6 |
| | 2 | Softener + 0.25% Cellulase of | 11.7 |
| | | SEQ ID NO: 4 | |
| | 3 | Softener | 11.2 |
| | 3 | Softener + 0.25% Cellulase of | 12.1 |
| | | SEQ ID NO: 4 | |
| | 4 | Softener | 11.6 |
| | 4 | Softener + 0.25% Cellulase of | 12.2 |
| | | SEQ ID NO: 4 | |
| | 5 | Softener | 11.4 |
| | 5 | Softener + 0.25% Cellulase of | 12.5 |
| | | SEQ ID NO: 4 | |
| W20A | 1 | Softener | 11 |
| | 1 | Softener + 0.25% Cellulase of | 11.9 |
| | | SEQ ID NO: 4 | |
| | 2 | Softener | 10.7 |
| | 2 | Softener + 0.25% Cellulase of | 11.5 |
| | | SEQ ID NO: 4 | |
| | 3 | Softener | 9.9 |
| | 3 | Softener + 0.25% Cellulase of | 11.2 |
| | | SEQ ID NO: 4 | |
| | 4 | Softener | 10.6 |
| | 4 | Softener + 0.25% Cellulase of | 11.8 |
| | | SEQ ID NO: 4 | |
| | 5 | Softener | 10.3 |
| | 5 | Softener + 0.25% Cellulase of | 11.4 |
| | | SEQ ID NO: 4 | |
| W30A | 1 | Softener | 11.4 |
| | 1 | Softener + 0.25% Cellulase of | 10.9 |
| | | SEQ ID NO: 4 | |
| | 2 | Softener | 11.1 |
| | 2 | Softener + 0.25% Cellulase of | 11.6 |
| | | SEQ ID NO: 4 | |
| | 3 | Softener | 11.1 |
| | 3 | Softener + 0.25% Cellulase of | 11.0 |
| | | SEQ ID NO: 4 | |
| | 4 | Softener | 11.5 |
| | 4 | Softener + 0.25% Cellulase of | 11.0 |
| | | SEQ ID NO: 4 | |
| | 5 | Softener | 10.8 |
| | 5 | Softener + 0.25% Cellulase of | 11.2 |
| | | SEQ ID NO: 4 | |

### Example 2—Water Absorption Improvement Evaluation

### Procedure for Preparing the Textiles

### Enzymes Used:

Cellulase A (SEQ ID NO: 1 and SEQ ID NO: 4), available from Novozymes A/S, Bagsvaerd, Denmark.

Cellulase B (SEQ ID NO: 3) available from Novozymes A/S, Bagsvaerd, Denmark.

Cellulase C (SEQ ID NO: 5) available from Danisco/Dupont.

### Detergent and Softener Used:

Detergent: wash liquor (100%) was prepared by dissolving 3.33 g/l of model detergent A in water with hardness 15 d H.

Softener: Ideel, Denmark (Aldi). Water, Cationic surfactants, Isopropyl alcohol, Calcium Chloride.

### Textiles Used:

W-10A (WFK standard cotton, 50×1 m), W-20A (WFK Polyester/Cotton 65/35%, 50×1 m), W-30A (WFK 100% Polyester, 50×1 m).

### Machine Used:

Miele Softronic WT2780.

### Washing Conditions:

Wash temperature: 40° C., short program (1 h 35 min), Water hardness: 15 dH, Water level (in wash): 13-14 L water (main wash), Ballast: Total of 3 kg, Spinning speed: 1600 rpm

Tumble drying: Miele Softronic WT2780.

Procedure: One sheet of each of the materials was added to the wash and 20 consecutive cycles were performed in the defined washing conditions with tumble drying in between each of the washes for the first 10 cycles, then after 15, 16 and 20. Water absorption was measured after the 20 cycles.

Results can be seen in Table 2 below.

### Procedure for Evaluating the Water Absortion of the Textiles:

Textiles were evaluated according to the vertical wicking test.
1. For each textile 5 2×20 cm stripes of textile were cut.
2. For each evaluation 20 ml of deionized water with food colorant (20 drops per 100 ml) were added in two 8 cm diameter beakers.
3. One stripe treated with softener and one stripe treated with softener with each of the cellulases were put vertically in one of the beakers (one in each beaker). One end was under the water level and the other end was attached to a support to ensure the stripes were absolutely vertical.
4. The height of the water level after 1 hour was measured.
5. The evaluation was done by quintuplicate (5 stripes).

TABLE 2
Results showing the water absorption improvement
when using a cellulase in a softener

| | | | Water level |
|---|---|---|---|
| | No. | | (cm) after |
| Textile | repetitions | | 1 h |
| W10A | 5 | Softener | 12.14 |
| | 5 | Softener + 0.40% Cellulase A | 13.10 |
| | 5 | Softener + 0.25% Cellulase B | 13.94 |
| | 5 | Softener + 0.25% Cellulase C | 11.86 |
| W20A | 5 | Softener | 17.0 |
| | 5 | Softener + 0.40% Cellulase A | 18.28 |
| | 5 | Softener + 0.25% Cellulase B | 16.52 |
| | 5 | Softener + 0.25% Cellulase C | 15.02 |
| W30A | 5 | Softener | 11.4 |
| | 5 | Softener + 0.40% Cellulase A | 14.96 |
| | 5 | Softener + 0.25% Cellulase B | 14.36 |
| | 5 | Softener + 0.25% Cellulase C | 14.42 |

### Example 3—Whiteness Improvement Evaluation

### Procedure for Preparing the Textiles

### Enzymes Used:

Cellulase of SEQ ID NO: 4 available from Novozymes A/S, Bagsvaerd, Denmark.

### Detergent and Softener Used:

Detergent: Ariel Colour & Style Powder, Denmark (P&G).

Softener: Doussy Summer Sun, Denmark (Lidl). Water, Cationic surfactants, Isopropyl alcohol, parfum, magnesium chloride, amyl cinnamal, butylphenyl methylpropional, colourant, dimethicone, benzisothiazolinona, metilisotiazolinona, sorbic acid, glutaral.

### Textiles Used:

W-10A (WFK standard cotton, 10×10 cm), EMPA 221 (Swissatest, Cotton fabric, cretonne, bleached, without optical brightener, 10×10 cm), H&M white towels (100% cotton).

Soil added: WFK greying swatch I (WFK).

### Machine Used:

Miele W5841.

### Washing Conditions:

Wash temperature: 40° C., short program (50 min of main wash), Water hardness: 15 dH, Water level (in wash): 15-16 L water (main wash), Ballast: Total of 4 kg.

### Tumble Dryer Used:

Miele PT 7501 EL (Inside diameter: 109 cm, deep bucket 53 cm).

### Drying Time:

30 min (equivalent to bone dry).

### Procedure for the Preparing the Textiles:

Six items of each of the textiles were washed for 10 cycles according to the defined washing conditions and tumble dried after each wash according to the defined tumble drying conditions. The same items were then washed 10 consecutive cycles in the same washing conditions without drying in between. In each of the cycles one WFK greying swatch was added to the wash. Three items of each were taken out of the wash after wash 15 and wash 20. The three items taken out after the 15^{th} and 20^{th} wash were tumble dried as described above. Once dried, the items were remission measured to determine the whiteness of the item. Results can be seen below in Tables 3 and 4.

Procedure for Evaluating the Whiteness of the Textiles:

The Remission value at wavelength 460 nm of the textiles was measured by duplicate using a standard Color Eye apparatus (Producer: Macbeth (USA, U.K., Germany), Supplier: Largo, Model: 370).

TABLE 3
Results of the whiteness improvement
when using a cellulase in a softener:

Average Rem 460 nm

| | H&M Towels - | H&M Towels - |
|---|---|---|
| | after 15 | after 20 |
| | cycles | cycles |
| Softener | 75.3 | 71.7 |
| Softener + 0.15% Cellulase of | 77.6 | 76.8 |
| SEQ ID NO: 4 | | |
| Softener + 0.25% Cellulase of | 79.2 | 77 |
| SEQ ID NO: 4 | | |

TABLE 4
Results of the whiteness improvement
when using a cellulase in a softener:

Average Rem 460 nm

| | W-10A - | EMPA 221 - |
|---|---|---|
| | after 15 | after 20 |
| | cycles | cycles |
| Softener | 68.8 | 69.9 |
| Softener + 0.15% Cellulase of | 71 | 72.1 |
| SEQ ID NO: 4 | | |
| Softener + 0.25% Cellulase of | 71.1 | 72.7 |
| SEQ ID NO: 4 | | |

### Example 4—Whiteness

### Procedure for Preparing the Textiles

### Enzymes Used:

Cellulase of SEQ ID NO: 4 available from Novozymes A/S, Bagsvaerd, Denmark.

### Detergent and Softener Used:

Detergent: Ariel Colour & Style Liquid, Denmark (P&G).

Softener: Ideel, Denmark (Aldi). Water, Cationic surfactants, Isopropyl alcohol, Calcium Chloride.

### Textiles Used:

W-10A (WFK standard cotton, 10×10 cm), W-20A (Polyester/Cotton 65/35, 10×10 cm), W-80A (WFK cotton knit, 10×10 cm), EMPA 210 (Swissatest, Cotton fabric, plain weave, bleached, without optical brightener, 10×10 cm), EMPA 211 (Swissatest, Cotton fabric, percale, bleached, without optical brightener, 10×10 cm), EMPA 213 (Swissatest, Polyester/cotton fabric, 65/35, bleached, without optical brightener, 10×10 xm), EMPA 221 (Swissatest, Cotton fabric, cretonne, bleached, without optical brightener, 10×10 cm), CFT CN-42 (Center for Testmaterials B.V., knitted cotton, 10×10 cm), CFT CN-11 (Center for Testmaterials B.V., Cotton Cretonne, bleached without optical brightner, woven, 10×10 cm), CFT PCN-01 (Center for Testmaterials B.V., Polyester/Cotton 65/35%, bleached w/o opt. br., woven).

### Soil Added:

WFK greying swatch I (supplier: WFK).

### Machine Used:

Miele Softronic W3241.

### Washing Conditions:

Wash temperature: 40° C., short program (1 h 35 min), Water hardness: 15 dH, Water level (in wash): 13-14 L water (main wash), Ballast: Total of 3 kg, Spinning speed: 1600 rpm Tumble drying: the built-in tumbledryer Miele Softtronic WT 2780 was used with the program “Skabskørt” (cupboard dry).

### Procedure for the Preparing the Textiles:

Six items of each of the textiles were washed for 10 cycles according to the defined washing conditions and tumble dried after each wash according to the defined tumble drying conditions. The same items were then washed 10 consecutive cycles in the same washing conditions without drying in between. In each of the cycles one WFK greying swatch was added to the wash. Three items of each were taken out of the wash after wash 15 and wash 20. The three items taken out after the 15^{th} and 20^{th} wash were tumble dried as described above. Once dried, the items were remission measured to determine the whiteness of the item. Results can be seen below in Table 5.

### Procedure for Evaluating the Whiteness of the Textiles:

The Remission value at wavelength 460 nm of the textiles is measured by duplicate using a standard Color Eye apparatus (Producer: Macbeth (USA, U.K., Germany), Supplier: Largo, Model: 370).

TABLE 5
Results of the whiteness improvement
when using a cellulase in a softener

| | Rem 460 nm | |
|---|---|---|
| | | Softener + 0.25% |
| | Softener | Cellulase of SEQ |
| Description of textile | solely | ID NO: 4 |
| Cotton fabric, plain weave, bleached, | 68 | 74 |
| without optical brightener (EMPA 210) | | |
| Cotton fabric, percale, bleached, without | 74 | 78 |
| optical brightener (EMPA 211) | | |
| Polyester/cotton fabric, 65/35, bleached, | 75 | 78 |
| without optical brightener (EMPA 213) | | |
| Cotton fabric, cretonne, bleached, without | 68 | 73 |
| optical brightener (EMPA 221 | | |
| Cotton interlock double jersey with optical | 70 | 75 |
| brightner (CFT CN-42) | | |
| Cotton Cretonne, bleached without optical | 67 | 74 |
| brightner, woven (CFT CN-11) | | |
| Polyester/Cotton 65/35%, bleached without | 70 | 74 |
| optical brightner, woven (CFT PCN-01) | | |
| Cotton Knit (WFK W80A) | 67 | 71 |
| Polyester/Cotton (65%/35%) (WFK W20A) | 71 | 76 |
| Standard Cotton (WFK W-10A) | 67 | 73 |

### Example 5—Whiteness

### Procedure for Preparing the Textiles

### Enzymes Used:

Cellulase A (SEQ ID NO: 1 and SEQ ID NO: 4), available from Novozymes A/S, Bagsvaerd, Denmark.

Cellulase B (SEQ ID NO: 3) available from Novozymes A/S, Bagsvaerd, Denmark.

Cellulase C (SEQ ID NO: 5) available from Danisco/Dupont.

### Detergent and Softener Used:

Detergent: wash liquor (100%) was prepared by dissolving 3.33 g/l of model detergent A in water with hardness 15 dH. Softener: Ideel, Denmark (Aldi). Water, Cationic surfactants, Isopropyl alcohol, Calcium.

### Textiles Used:

EMPA 210 (Swissatest, Cotton fabric, plain weave, bleached, without optical brightener, 10×10 cm), EMPA 211 (Swissatest, Cotton fabric, percale, bleached, without optical brightener, 10×10 cm), EMPA 213 (Swissatest, Polyester/cotton fabric, 65/35, bleached, without optical brightener, 10×10 xm), EMPA 221 (Swissatest, Cotton fabric, cretonne, bleached, without optical brightener, 10×10 cm), CFT CN-42 (Center for Testmaterials B.V., knitted cotton, 10×10 cm), CFT CN-11 (Center for Testmaterials B.V., Cotton Cretonne, bleached without optical brightner, woven, 10×10 cm), CFT PCN-01 (Center for Testmaterials B.V., Polyester/Cotton 65/35%, bleached w/o opt. br., woven).

### Soil Added:

WFK greying swatch I (supplier: WFK).

### Machine Used:

Miele Softronic WT2780.

### Washing Conditions:

Wash temperature: 40° C., short program (1 h 35 min), Water hardness: 15 dH, Water level (in wash): 13-14 L water (main wash), Ballast: Total of 3 kg, Spinning speed: 1600 rpm Tumble drying: The built-in tumbledryer Miele Softtronic WT 2780.

### Procedure for the Preparing the Textiles:

Six items of each of the textiles were washed for 10 cycles according to the defined washing conditions and tumble dried after each wash according to the defined tumble drying conditions. The same items were then washed 10 consecutive cycles in the same washing conditions without drying in between. In each of the cycles one WFK greying swatch was added to the wash. Four items of each were taken out of the wash after wash 16 and wash 20. The four items taken out after the 16^{th} and 20^{th} wash were tumble dried as described above. Once dried, the items were remission measured to determine the whiteness of the item. Results can be seen below in Tables 6 and 7.

### Procedure for Evaluating the Whiteness of the Textiles:

The Remission value at wavelength 460 nm of the textiles is measured by duplicate using a standard Color Eye apparatus (Producer: Macbeth (USA, U.K., Germany), Supplier: Largo, Model: 370).

TABLE 6
Results of the whiteness improvement when using a cellulase in a softener, 16 cycles

| | Rem 460 nm | | | |
|---|---|---|---|---|
| | | Softener + | Softener + | Softener + |
| | Softener | 0.25% | 0.25% | 0.25% |
| Description of textile | solely | Cellulase A | Cellulase B | Cellulase C |
| Cotton fabric, plain weave, bleached, | 69.1 | 79.4 | 78.2 | 77.3 |
| without optical brightener (EMPA 210) | | | | |
| Cotton fabric, percale, bleached, | 72.6 | 84.2 | 84.5 | 81.6 |
| without optical brightener (EMPA 211) | | | | |
| Polyester/cotton fabric, 65/35, | 76.8 | 83.6 | 83.2 | 81.9 |
| bleached, without optical brightener | | | | |
| (EMPA 213) | | | | |
| Cotton fabric, cretonne, bleached, | 72.0 | 82.6 | 81.3 | 80.8 |
| without optical brightener (EMPA 221 | | | | |
| Cotton interlock double jersey with | 75.2 | 86.8 | 85.9 | 85.5 |
| optical brightner (CFT CN-42) | | | | |
| Cotton Cretonne, bleached without | 72.0 | 82.7 | 81.9 | 81.4 |
| optical brightner, woven (CFT CN-11) | | | | |
| Polyester/Cotton 65/35%, bleached | 74.6 | 81.1 | 81.3 | 80.1 |
| without optical brightner, woven (CFT | | | | |
| PCN-01) | | | | |
| Cotton Knit (WFK W80A) | 70.7 | 81.2 | 80.7 | 80.5 |

TABLE 7
Results of the whiteness improvement when using a cellulase in a softener, 20 cycles

| | Rem 460 nm | | | |
|---|---|---|---|---|
| | | Softener + | Softener + | Softener + |
| | Softener | 0.25% | 0.25% | 0.25% |
| Description of textile | solely | Cellulase A | Cellulase B | Cellulase C |
| Cotton fabric, plain weave, bleached, | 65.5 | 78.5 | 75.6 | 76.2 |
| without optical brightener (EMPA 210) | | | | |
| Cotton fabric, percale, bleached, | 70.45 | 83.5 | 82.0 | 79.8 |
| without optical brightener (EMPA 211) | | | | |
| Polyester/cotton fabric, 65/35, | 54.3 | 82.5 | 81.7 | 80.3 |
| bleached, without optical brightener | | | | |
| (EMPA 213) | | | | |
| Cotton fabric, cretonne, bleached, | 66.0 | 79.6 | 78.7 | 78.7 |
| without optical brightener (EMPA 221 | | | | |
| Cotton interlock double jersey with | 69.4 | 84.9 | 83.3 | 84.0 |
| optical brightner (CFT CN-42) | | | | |
| Cotton Cretonne, bleached without | 66.8 | 80.2 | 80.6 | 79.3 |
| optical brightner, woven (CFT CN-11) | | | | |
| Polyester/Cotton 65/35%, bleached | 69.5 | 79.1 | 79.6 | 77.7 |
| without optical brightner, woven (CFT | | | | |
| PCN-01) | | | | |
| Cotton Knit (WFK W80A) | 66.4 | 80.3 | 78.9 | 79.3 |

1-14. (canceled)

15. A softener composition for use in improving water absorption and/or whiteness of a textile, wherein said softener composition comprises a family GH 45 cellulase, preferably a cellulase having at least 60% sequence identity to SEQ ID NO: 1, 2, 3, 4 or 5.

16. A method for improving water absorption of a textile comprising contacting a surface, such as a fabric surface, with an enzyme and a softener during a rinse cycle such as a laundry rinse cycle in a washing machine.

17. A method for improving whiteness of a textile comprising contacting a surface, such as a fabric surface, with an enzyme and a softener during a rinse cycle such as a launder rinse cycle in a washing machine.

18. The method according to claim 16, wherein said enzyme is a family GH45 cellulase.

19. The method according to claim 16, wherein said enzyme is a cellulase having at least 60% sequence identity to SEQ ID NO: 1, 2, 3, 4, or 5.

20. The method according to claim 16, wherein said softener comprises cationic surfactants.

21. The method according to claim 16, wherein said softener has a pH of at least 2.0.

22. The method according to claim 16, wherein said textile has been pre-washed in a laundering process.

23. The method according to claim 16, wherein said textile is cotton, polyester or a mixture thereof.

24. The method according to claim 23, wherein said mixture consists of at least 50% polyester and at least 20% cotton.

25. The method according to claim 22, wherein said pre-washing has been done at a temperature of at least 5° C., such as at least 10° C., at least 15° C., at least 20° C., at least 25° C., at least 30° C., at least 35° C., at least 40° C., at least 45° C., or at least 50° C.

26. The method according to claim 16, wherein said enzyme is added in a concentration of at least 0.01% of said softener.

27. The method according to claim 17, wherein said textile is cotton.

28. The method according to claim 22, wherein said pre-washing has been done at a temperature of at least 5° C., such as at least 10° C., at least 15° C., at least 20° C., at least 25° C., at least 30° C., at least 35° C., at least 40° C., at least 45° C., or at least 50° C.

29. The method according to claim 17, wherein said enzyme is added in a concentration of at least 0.01% of said softener.
